(19) United States
(12) Patent Application Publication
(10) Pub. No.: US 2015/0045656 A1
(43) Pub. Date: Feb. 12, 2015

(54) SURGICAL NAVIGATION SYSTEM

(71) Applicants: Industry-University Cooperation Foundation Hanyang University Erica Campus, Ansan, Gyeonggi-do (KR); KOH Young Technology Inc., Geumcheon, gu, Seoul (KR)

(72) Inventors: Hyun-Soo Yoon, Ansan-si (KR); Byung-Ju Yi, Bucheon-si (KR)

(73) Assignees: INDUSTRY-UNIVERSITY COOPERATION FOUNDATION HANYANG UNIVERSITY ERICA CAMPUS, Ansan-si, Gyeonggi-do (KR); KOH YOUNG TECHNOLOGY INC., seoul (KR)

(21) Appl. No.: 14/007,505

(22) PCT Filed: Apr. 22, 2013
(86) PCT No.: PCT/KR2013/003375

§ 371 Date: Sep. 25, 2013

(30) Foreign Application Priority Data
Apr. 25, 2012 (KR) 10-2012-0043471
Apr. 25, 2012 (KR) 10-2012-0043482

(51) Int. Cl. A61B 19/00; A61B 17/24; A61B 17/00

(52) U.S. Cl. CPC A61B 19/5244; A61B 17/00234; A61B 17/24; A61B 19/54; A61B 2017/00314; A61B 2019/2211; A61B 2019/5445; A61B 2019/5483
USPC 600/424

(57) ABSTRACT

An surgical navigation system to provide more precise navigation function during endoscopic surgery in otolaryngology such as paranasal sinuses including large curved areas. The surgical navigation system comprises a bendable surgical instrument containing plurality of markers to generate energy, a master device coupled to the bendable surgical instrument to activate the markers, a tracking sensor to detect energy generated from the markers; and a processor coupled to the tracking sensor and the bendable surgical instrument, to determine a position of the energy which is generated from the markers detected by the tracking sensor, and to trace confirmed markers wherein the confirmed markers are matched between the position of the energy detected by the tracking sensor with a predetermined marker stored in the processor.

### TECHNICAL FIELD

Exemplary embodiments of the present invention relate to a surgical navigation system. More particularly, exemplary embodiments of the present invention relate to a surgical navigation system for an otolaryngology endoscopic surgery like paranasal sinuses

### BACKGROUND ART

In general, an endoscopic surgery proceeds a surgery while watching an affected area of a patient through an image taken by an endoscope, in which surgical instruments and an endoscope camera is installed through a small hole, without making large incision.

Endoscopic surgery, started from laparoscopic surgery, has an advantage of a fast recovery time of surgical patients compared to open surgery since the incision is small and less bleeding.

Recently, endoscopic surgery system is technically developed enough to be used almost all type of open surgery as well as other field of medicine.

Meanwhile, otolaryngology, a branch of medical science, handles anatomy, physiology, and pathology of ear, nose, pharynx, and larynx, and applies an endoscope to various types of surgeries and treatments. During the endoscopic surgery, conventional probes with straight shape or curved fixed shape are used.

Such conventional probes have a difficulty to navigate precisely since there are many areas impossible to access during diagnosing and proceduring.

Specifically, using such conventional probes during diagnosing and proceduring in otolaryngology, is hard to navigate precisely as there are many areas impossible to access though the probe such as paranasal sinuses

### DISCLOSURE

### Technical Problem

Therefore, the aim of the present invention is to provide a surgical navigation system with more precise navigation function in an endoscopic surgery in otolaryngology such as paranasal sinuses

### Technical Solution

According to an embodiment of the present invention, the surgical navigation system can trace the position of the probe through the processor. In addition, a user can bend the probe to a desired direction and angle and the position and curvature information of the curved area, obtained from the bend sensor, can be transmitted to the processor through the communication module. Accordingly, it is possible to provide exact navigating function where various curved body parts, such as paranasal sinuses, are positioned in otolaryngology surgery and therefore, it is effective to proceed the surgery more safely as well as be able to be extended to a further range of endoscopic surgery.

In one embodiment of the present invention, a surgical navigation system includes a bendable surgical instrument including a plurality of markers generating an energy, a master device coupled to the instruments activating the markers, a tracking sensor detecting the energy generated by the markers, and a processor coupled to the surgical instrument and the sensor determining a position of energy detected by the tracking sensor, and tracing confirmed markers mapped to the position of the energy detected by the tracking sensor.

In some instance, the bendable surgical instrument may include a probe having a bendable section and an operator including the markers, wherein the markers generate energy activated by the master device.

In some instance, the bendable section may include a plurality of cylinders separated in a regular interval, a plurality of operating tools passing through the cylinders and coupled to the operator to bend the cylinders to a desired direction at the same time, and a plurality of elastic members interposed between the cylinders to support elasticity between the adjacent cylinders. Herein, the operating tools may be wire, and the elastic member may be coil spring.

Meanwhile, the bendable surgical instrument may further include at least one bend sensor to obtain a position and curvature information.

In addition, the bendable surgical instrument may further include a communication module coupled to the bend sensor and the processor to transmit the obtained information from the bend sensor.

### Advantageous Effects

Thus, according to an embodiment of the present invention, the surgical navigation system can trace the position of the probe through the processor. In addition, a user can bend the probe to a desired direction and angle. Also the position and curvature information of the curved area which are obtained from the bend sensor, can be transmitted to the processor through the communication module. Accordingly, it is possible to provide exact navigating function where various curved body parts, such as paranasal sinuses, are positioned in otolaryngology surgery and therefore, it is effective to proceed the surgery more safely as well as be able to be extended to a further range of endoscopic surgery.

### DESCRIPTION OF DRAWINGS

FIG. 1 is a schematic view of a surgical navigation system according to an embodiment of the present invention;

FIG. 2 is a diagram showing a coordinate system of the surgical navigation system

FIG. 3 is a perspective view of bendable surgical instrument of according to an embodiment of the present invention;

FIG. 4 is an enlarged view of FIG. 3A;

FIG. 5 is a perspective view of a cylinder;

FIG. 6 is a block diagram to explain the bendable surgical instrument according to an embodiment of the present invention.

### MODE FOR INVENTION

The present invention is described more fully hereinafter with reference to the accompanying drawings, in which example embodiments of the present invention are shown. The present invention may, however, be embodied in many different forms and should not be construed as limited to the example embodiments set forth herein. Rather, these example embodiments are provided so that this disclosure will be thorough and complete, and will fully convey the scope of the present invention to those skilled in the art. In the drawings, the sizes and relative sizes of layers and regions may be exaggerated for clarity.

It will be understood that, although the terms first, second, third etc. may be used herein to describe various elements, components, regions, layers and/or sections, these elements, components, and/or sections should not be limited by these terms. These terms are only used to distinguish one element, component, region, layer or section from another region, layer or section. Thus, a first element, component, or section discussed below could be termed a second element, component, or section without departing from the teachings of the present invention.

The terminology used herein is for the purpose of describing particular example embodiments only and is not intended to be limiting of the present invention. As used herein, the singular forms “a,” “an” and “the” are intended to include the plural forms as well, unless the context clearly indicates otherwise. It will be further understood that the terms “comprises” and/or “comprising,” when used in this specification, specify the presence of stated features, integers, steps, operations, elements, and/or components, but do not preclude the presence or addition of one or more other features, integers, steps, operations, elements, components, and/or groups thereof.

Unless otherwise defined, all terms (including technical and scientific terms) used herein have the same meaning as commonly understood by one of ordinary skill in the art to which this invention belongs. It will be further understood that terms, such as those defined in commonly used dictionaries, should be interpreted as having a meaning that is consistent with their meaning in the context of the relevant art and will not be interpreted in an idealized or overly formal sense unless expressly so defined herein.

For convenience, same numerals are used for identical or similar elements of an apparatus of cutting a tempered substrate and the conventional one.

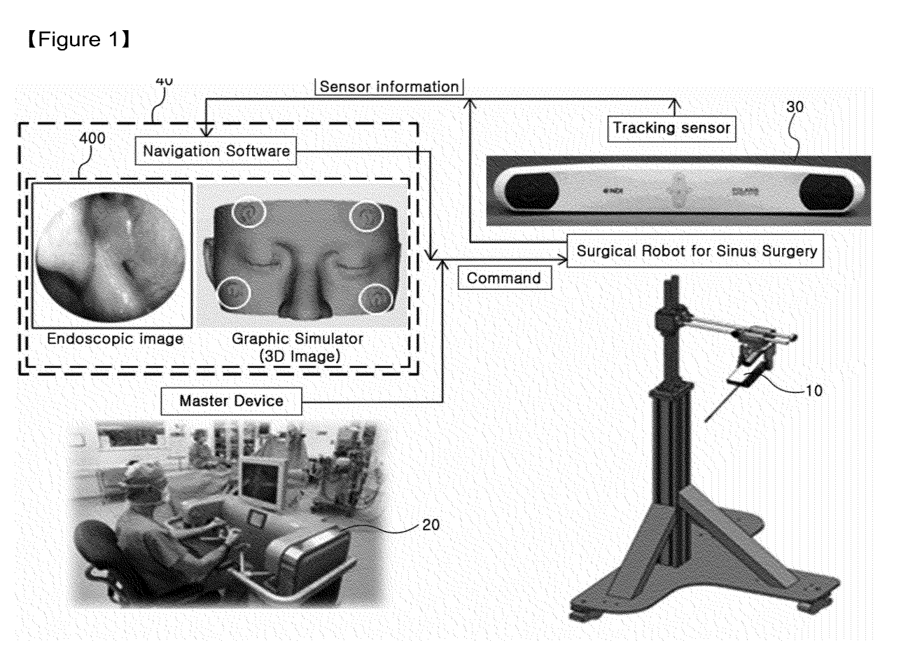

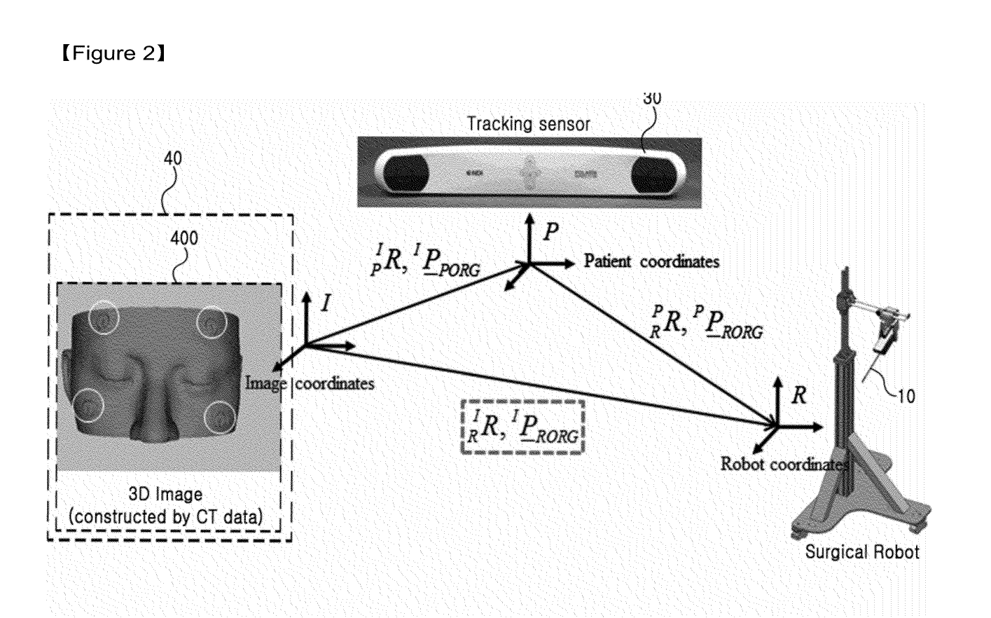

FIG. 1 is a schematic view of a surgical navigation system according to the present invention and FIG. 2 is a diagram showing the coordinate system of the surgical navigation system.

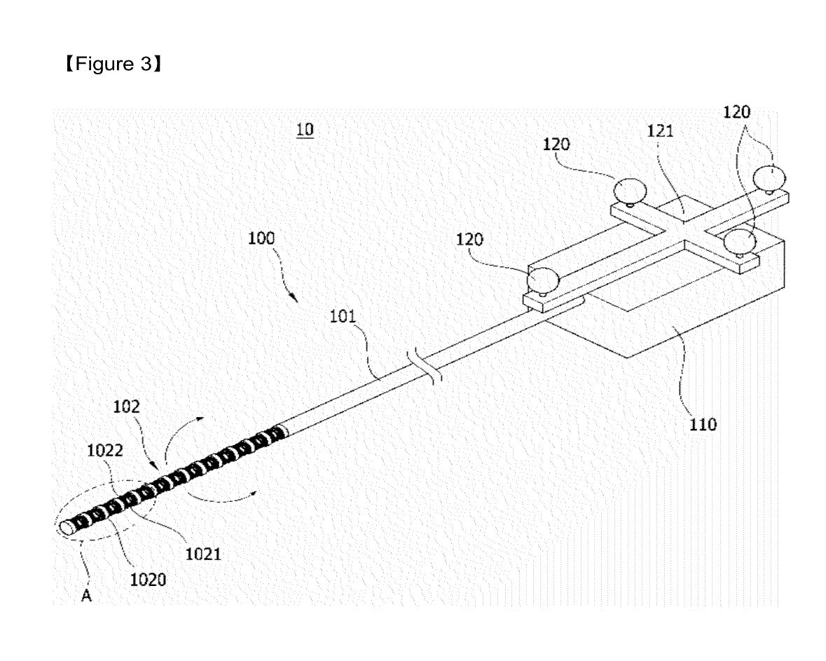

Referring to FIGS. 1 and 2, a surgical navigation system according to the present invention, includes a bendable surgical instrument 10, a master device 20, a tracking sensor 30, and a processor 40. The bendable surgical instrument 10 includes a plurality of markers 120(Refer FIG. 3) generating energy. The master device, coupled to the bendable surgical instrument, activates the markers to generate energy. The tracking sensor 30 detects energy generated from the markers 120. Meanwhile, the tracking sensor 30 also detects energy generated from markers(not shown) attached to a patient.

The processor 40, coupled to the tracking sensor 30 and the bendable surgical instrument, determines a position of the energy which is generated from the markers and detected by the tracking sensor 30, and then, traces confirmed markers matched with previously set makers of the processor 40, which are corresponding to energy position of the detected markers. In other words, since the position of the markers are predetermined in the processor 40, the processor 40, after determining the position of the energy generated from the markers that are detected by the tracking sensor 30, traces confirmed markers that are matched with the position of the marker to the predetermined position stored in the processor. Meanwhile, the processor 40 may further include a display to render an image. The image may include endoscopic image and a graphic simulation. For example, the graphic simulation may be 2D or 3D images generated from CT image data.

As described in FIG. 2, the processor 40 may match the coordinate of 2D or 3D images generated from graphic simulation data shown in the display 400, the coordinate of markers(not shown) attached to the patient which are determined by detecting generated energy by tracking sensor 30, and the coordinate of markers disposed in the bendable surgical instrument which are determined by detecting generated energy from the markers 120(Refer FIG. 3).

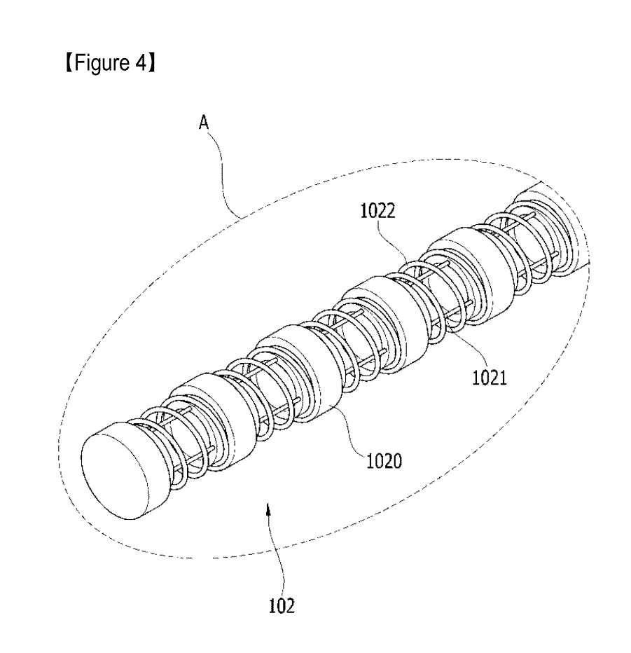

The following is a configuration of the bendable surgical instrument according to one embodiment of the present invention with reference to FIGS. 3 to 6.

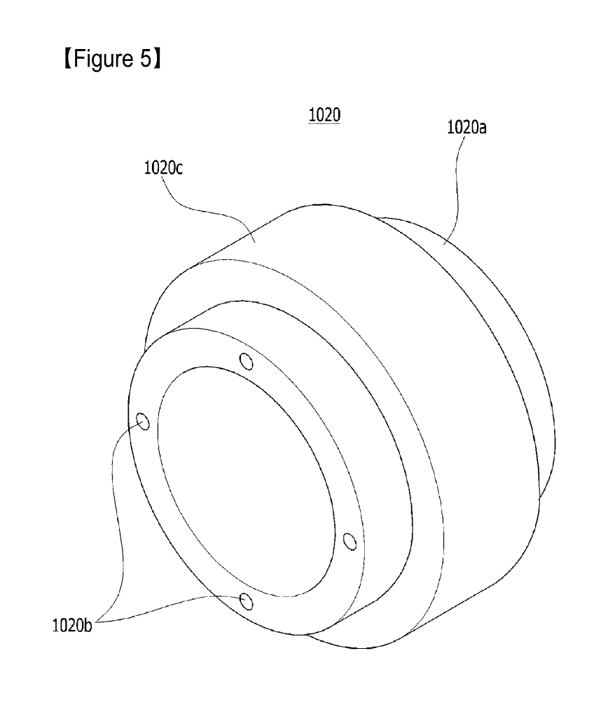

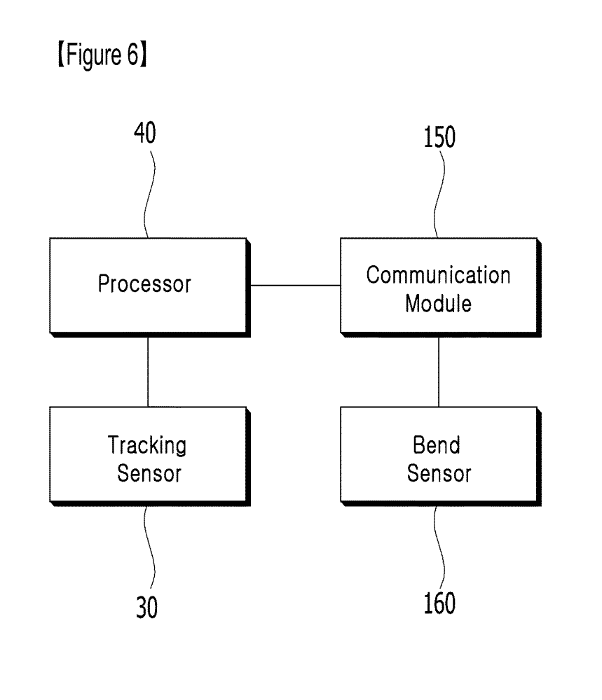

FIG. 3 is a perspective view of a bendable surgical instrument of according to the present invention, FIG. 5 is a perspective view of a cylinder, and FIG. 6 is a block diagram to explain the bendable surgical instruments according to the present invention.

Referring to FIGS. 3 to 6, the bendable surgical instrument 10 includes a probe 100, an operator 110, and a plurality of markers 120.

The probe 100 contains a fixed section 101 and a bendable section 102. The fixed section 101 is coupled to the operator 110. It is preferable to couple the fixed section 101 to the operator 110 such that the fixed section 101 may rotate with respect to the operator 110. In a detailed description, the bendable section 102 contains a plurality of cylinders 1020, a plurality of operating tools 1021, and a plurality of elastic members 1022. The plurality of cylinders 1020 is aligned in line in a regular interval. A cylinder of the plurality of cylinders 120, which is disposed at one side or opposite side, is coupled to the fixed section 101. The cylinder 1020 includes a body 1020a, a plurality of penetrating holes 1020b, and a supporting jaw 1020c. The body 1020a has a pipe shaped with a center hole. The penetrating holes 1020b pass through the body 1020a. In other words, the penetrating holes 1020b pass through the body from one side to the other side direction. The penetrating holes 1020b penetrate the body 1020a in a regular interval when viewing the cylinder 1020 in an axial direction. It is preferable to maintain the interval in 90 degrees in an axial direction. The Supporting jaw 1020c supports the elastic member 1022 surrounding the outer peripheral surface of the body 2010a.

The operating tools 1021 couple the operator 110 and the cylinders 1020 which are aligned in a regular interval, and bend the cylinders 1020 to a desired direction at the same time. More specifically, the operating tools 1021 pass through the penetrating holes 1020b which are aligned in a regular interval and coupled the cylinders 1020 to one another, penetrate to the internal of the fixed section 101 and are coupled to the operator to bend the cylinders to a desired direction. In some instance, the operating tools 1021 may be wires.

The plurality of elastic members 1022 is interposed between the cylinders 1020 and provides elasticity between adjacent cylinders 1020. In other words, the plurality of elastic members 1022 are interposed between the cylinders 1020 to provide elasticity between adjacent cylinders 1020, so that the both side of the cylinders 1020 are supported by the supporting jaw 1020c.

The operator 110 is coupled to the probe 100 and activates the probe 100. More detail, the operator 110 is coupled to the probe 100 such that the probe 100 may rotate with respect to the operator 110. Meanwhile, the operator 110 contains a rotating tool(not shown) to be coupled with and rotate the probe 100. The plurality of winding tools(not shown) is disposed in the operator 110 and wraps the operating tools. Therefore, the plurality of winding tools is rotated, by pulling and unwrapping the plurality of wires, and bends the bendable section of the probe 102 to a desired direction and angle.

The tracking sensor 30 detects the energy generated by the markers 120 activated by the operator. For example, the markers 120 are disposed in a stage of the operator 110. The stage should be criss-cross shaped form, and the four markers are disposed at the end of each stage. The plurality of markers may be self-luminous, reflective to reflect the light, or in combination of both in a certain proportion. Also the markers 120 can use electromagnetic. The markers 120 mentioned above, may self-generate light to release electromagnetic, or reflect the light from lighting system (not shown). The tracking sensor 30 detects energy from released, reflected light or released electromagnetic generated from the marker, and transmit to the processor 40. After then, the processor 40 matches the energy generated from the marker 120 with the predetermined location of the marker 120 to trace the confirmed markers 120.

The bendable surgical instrument 10 according to the present invention may further include a bend sensor 160. The bend sensor is disposed in the probe 100 and may obtain location and curvature information of the bended area 102.

The bendable surgical instrument 10 according to the present invention may further include a communication module 150. The communication module, coupled to the bend sensor 160 and the processor 40, receives location and curvature information from the bend sensor, and transmit to the processor 40.

Thus, as the surgical navigation system according to the present invention can provide more precise navigating function, it is possible to proceed the endoscopic surgery in otolaryngology, such as paranasal sinusesm, more safely as well as be able to be extended to a further range of endoscopic surgery in otolaryngology.

The following is a operating method of the bendable surgical instrument according to one embodiment of the present invention with reference to FIGS. 3 to 6.

Referring FIGS. 3 to 6, the bendable surgical instrument 10 according to the present invention bends the bendable section 102 of the probe 100 to a desired angle and direction by activating the plurality of operating tools 1021 of the operator 110. More specifically, selecting at least one winding tool(not shown) disposed in the operator 110, and activating the selected winding tool to wrap the at least one selected operating tool 1021. When the operating tool is wrapped to the winding tool as above, the bendable section 102 are pulled by the operating tool, so that the bendable section 102 of the probe is bent to a pulling direction. Hereat, the operating tool, facing the operating tool wrapped to the winding tool, may easily bend the curves section 102 of the probe 100 as the winding tool is unwrapped .

Meanwhile, the elastic member 1022 interposed between the plurality of cylinders of the bendable section 102 is pressed by the adjacent cylinder, and is curved and compressed to the direction in which the operating tool 1021 is disposed. And, by unwrapping the operating tool 1021 wrapped by winding tool, the cylinders 1020 curved to the operating tool direction are restored to a straight line(original) state by a force of restitution of the elastic member. Hereat, the operating tool, facing with the unwrapping operating tool by the winding tool, pulls the bendable section 102 wrapped by the winding tool the to the opposite direction to the curved direction, so that the elastic member can easily and promptly restore the bendable section to the straight line(original) state.

Thus, according to the present invention, the surgical instrument 10 can easily bend the bendable section 102 of the probe 100 to a desired direction and angle, so that it is possible to access easily to a inaccessible areas during diagnosing and proceduring the patient, which is not possible using conventional probes.

At the same time, the bend sensor 160 disposed in the probe 100 detects the position and the curvature information of the bendable section 102, the communication module 150 receives the position and the curvature information of the bendable section 102 from the bend sensor and transmit to the processor 40, so that the processor 40 can identify the exact position and curvature information of the bendable section 102.

Also, the markers 120 are self-luminous, releases an electromagnetic, or reflects light generated from the lighting system(not shown). The tracking sensor 30, coupled to the processor 40, detects the light released from the marker 120 or reflected by the markers, and sends to the processor 40 to identify the position of the markers 120. The processor 40 can identify the exact position of the probe 100 by combining distance between the pre-stored markers and separated probe 100, and the position and the curvature information of the bendable section 102.

As described above, the bendable surgical instrument 10 according to the present invention, includes a bendable section 102 at the end of the probe 100 so that a user can bend to a desired direction and angle, accessing inaccessible areas during diagnosing and proceduring the patient which is not possible using conventional probes. As the processor can track the position of the probe 100, the position of the end of the bendable section 102, and the curvature information detected by the bend sensor at the same time, it is possible to provide more precise navigation function.

It will be apparent to those skilled in the art that various modifications and variation can be made in the present invention without departing from the spirit or scope of the invention. Thus, it is intended that the present invention cover the modifications and variations of this invention provided they come within the scope of the appended claims and their equivalents.

1. A surgical navigation system comprising:
a bendable surgical instrument including a plurality of markers which generates energy;
a master device being coupled to the bendable surgical instrument and activating the markers;
a tracking sensor detecting energy generated from the markers; and
a processor being coupled to the tracking sensor and the bendable surgical instrument, determining a position of the energy which is generated from the markers and detected by the tracking sensor, and to tracing confirmed markers with previously set markers of the processor, which are corresponding to energy position of the detected markers.

2. The surgical navigation system of claim 1, wherein the bendable surgical instrument comprises:
a probe including a bendable section;
an operator being coupled to the probe, and including the plurality of markers activated by the master device to generate energy.

3. The surgical navigation system of claim 2, wherein the bendable section comprises:
a plurality of cylinders being separated in a regular interval;
a plurality of operating tools passing through the cylinders, being coupled to the operator, and bending the cylinders to a desired direction; and
a plurality of elastic members being interposed between adjacent cylinders and supporting elasticity between the cylinders.

4. The surgical navigation system of claim 3, wherein the operating tools comprises wires.

5. The surgical navigation system of claim 3, wherein the elastic members comprise coil springs.

6. The surgical navigation system of claim 2, wherein the bendable surgical instrument further comprises a bend sensor being disposed in the probe and obtaining a position and curvature information of the bendable section.

7. The surgical navigation system of claim 6, wherein the bendable surgical instrument further comprises a communication module being disposed in the probe and being coupled to the bend sensor and the processor, and transmitting the position and the curvature information to the processor.
